SEQUENCE LISTING (12 sequences)

SEQ ID NO: 1 - DNA, 21 bp, Artificial Sequence
  Primer
ctctcctatctctgctctgaa

SEQ ID NO: 2 - DNA, 20 bp, Artificial Sequence
  Primer
actcacttttccgaggctga

SEQ ID NO: 3 - DNA, 20 bp, Artificial Sequence
  Primer
cctctgtcaacaaaggtgat

SEQ ID NO: 4 - DNA, 20 bp, Artificial Sequence
  Primer
gtgctggcatcagagtgtgt

SEQ ID NO: 5 - DNA, 21 bp, Artificial Sequence
  Primer
actgacatggaacaggggaag

SEQ ID NO: 6 - DNA, 24 bp, Artificial Sequence
  Primer
ccagctctgaagtagggaacatat

SEQ ID NO: 7 - DNA, 22 bp, Artificial Sequence
  Primer
ggctatcattcttcttcaaggt

SEQ ID NO: 8 - DNA, 22 bp, Artificial Sequence
  Primer
cctctcttcagccatttaagta

SEQ ID NO: 9 - DNA, 27 bp, Artificial Sequence
  Probe
aataagttcagccctccatgtcacact

SEQ ID NO: 10 - DNA, 26 bp, Artificial Sequence
  Probe
tgttttccatcctggggagtgtttca

SEQ ID NO: 11 - DNA, 34 bp, Artificial Sequence
  Probe
ggctgaaggttagggataccaatattcctgtctc

SEQ ID NO: 12 - DNA, 30 bp, Artificial Sequence
  Probe
ctagtgatgggctcttcccttgagcccttc

(19) United States
(12) Patent Application Publication
(10) Pub. No.: US 2008/0112934 A1
(43) Pub. Date: May 15, 2008

(54) Methods To Identify, Prepare, And Use Naive T Cell Recent Thymic Emigrants

(76) Inventors: Rafick-Pierre Sekaly, Saint-Laurent (CA); Lionel G. Filion, Ottawa (CA); Marie-Lise Dion, Montreal (CA); Remi Cheynier, Rambouillet (FR); Hugues Thiebot, Issy-les-Moulineaux (FR)

Correspondence Address: GOUDREAU GAGE DUBUC, 2000 MCGILL COLLEGE, SUITE 2200, MONTREAL, QC, H3A 3H3, omitted

(21) Appl. No.: 11/720,124

(22) PCT Filed: Nov. 24, 2005
(86) PCT No.: PCT/CA05/01785

§ 371 Date: May 24, 2007

Related U.S. Application Data
(60) Provisional application No. 60/630,195, filed on Nov. 24, 2004.

(51) Int. Cl. A61K 35/55; G01N 33/567; C12N 5/08

(52) U.S. Cl. 424/93.7; 435/7.2; 435/366

(57) ABSTRACT

A method of purifying a subpopulation of naive T cells that have recently emigrated from the thymus, comprising (a) contacting a biological sample susceptible of containing T cells with at least four different ligands, namely ligands directed to the markers CD3, CD4, CD45RA, and CD31; (b) sorting the cells so as to recover T cells having a phenotype comprising at least the following four markers CD3⁺, CD4⁺, CD45RA⁺ and CD31^{hi}, whereby a subpopulation of naive T cells is purified.

### CROSS REFERENCE TO RELATED APPLICATIONS

This application claims priority on U.S. provisional application No. 60/630,195 filed on Nov. 24, 2004. All documents above are herein incorporated by reference.

### FIELD OF THE INVENTION

The present invention relates to methods to identify, quantify and purify recent thymic emigrants (RTEs); method to assess thymic function; kits for use in these methods; and purified recent thymic emigrants. More specifically, the present invention is concerned with a more precise phenotype for RTEs.

### BACKGROUND OF THE INVENTION

The thymus is the main source of T cells replenishing the naive T cell pool with a diverse T cell repertoire. These newly produced cells are exported to the periphery as recent thymic emigrants (RTEs), a sub-compartment of antigen-naive T cells. The exact nature of RTEs remains unclear and determining their phenotype is key in understanding their physiology and overall behavior in pathological settings, such as HIV. In such a disorder, where total T cells numbers are greatly reduced, the rebuilding of the T cell pool must include the generation of new T cells by the thymus and monitoring this immune reconstitution necessitates a reliable identification of RTEs.

The quantification of T cell receptor (TCR) excision circles (TRECs), an intrinsic marker for RTEs, has permitted the assessment of thymic function in humans. TRECs are by-products of TCR rearrangement, a process that mainly occurs within the thymus. More specifically, the sjTREC (signal jointTREC)¹, the most commonly used TREC in overall thymic output quantification, results from the excision of the TCRD (TCRδ) locus within the TCRA (TCRα) locus, a rearrangement that occurs in approximatively 66% of thymocytes. These circular episomal DNA molecules are maintained in the newly exported T cells and diluted upon homeostatic and TCR engagement-induced proliferation. TRECs, as they do not duplicate, are diluted among dividing cells, reducing the overall frequency exponentially in further differentiated cells. Indeed, higher TREC levels have been observed in CD45RA+CD62L+ naive T cells when compared to non-naive CD45RO+CD62L− T cells². However, as TRECs are DNA molecules, their PCR-based quantification implies the lysis of cells, rendering sorting and functional characterization of RTE quite difficult.

To overcome this limitation, this group sought to identify a series of cell surface markers in peripheral blood for which cells are enriched in TRECs levels, closest to exiting thymocyte TREC levels, thus defining the RTE phenotype. To do so, CD31, or PECAM-1 (platelet endothelial cell adhesion molecule-1) was added to the naive phenotype (CD45RA+CD62L+ and/or CD27+). This molecule seemed a potentially good candidate for defining RTE because CD31 is lost upon TCR engagement and, concomitant to thymic function, the % of CD31⁺ cells within the CD45RA⁺ decreases with age³. Most importantly, it has been reported that CD31 distinguishes two naive T cell subsets (defined by this author as CD45RA+ cells), CD31⁺ cells being 8-fold enriched in TRECs as compared to CD31−. Although, this group considered that CD31+ cells could be thymic naïve T cells, sufficient data was not provided to clearly qualify these cells as RTE ³. Indeed, this recent study failed to provide convincing data that compared TREC levels within CD31+ cells to levels found within the late stage thymocytes. Furthermore, the naive T cells were lightly defined, namely with the phenotype CD45RA⁺ CD4⁺T cells. In healthy individuals, selecting CD45RA⁺ T cells identifies 95-98% of naive cells, as there are very few activated T cells present. However, in pathological settings such as HIV, where immune activation is greatly enhanced, further definition of naive T cells, using CD27 and/or CD62L is needed because a subset of activated cells also expose the CD45RA antigen. In addition, the many steps used by Kimmig et al. to purify the thymic naive T cells may have introduced biases and limited the purity of the isolated population.

There is still a need for obtaining purified naive T cells representative of exiting thymic cells.

The present invention seeks to meet these needs and other needs.

The present description refers to a number of documents, the content of which is herein incorporated by reference in their entirety.

### SUMMARY OF THE INVENTION

This invention relates to a method making use of ligands to at least the following naive T cell markers (CD3⁺CD4⁺CD45RA⁺), along with ligands to CD31 enabling the identification, sorting and purification of naive T cells (CD4 T cells having a RTE phenotype) in individuals.

More specifically, in accordance with a first aspect of the present invention, there is provided a method of purifying a subpopulation of naive T cells that have recently emigrated from the thymus, comprising (a) contacting a biological sample susceptible of containing T cells with at least four different ligands, namely ligands directed to the markers CD3, CD4, CD45RA, and CD31; (b) sorting the cells so as to recover T cells having a phenotype comprising at least the following four markers CD3⁺, CD4⁺, CD45RA⁺ and CD31^{hi}, whereby a subpopulation of naive T cells is purified. In a specific embodiment of the method, said contacting step comprises contacting the sample with at least two additional ligands, namely ligands directed to the markers CD27⁺ and CD62L⁺ or CCR7⁺. In an other specific embodiment of the method, said at least two additional ligands comprise a CD62L ligand, and wherein said sample has not been frozen. In an other specific embodiment of the method, said at least two additional ligands comprise a CCR7 ligand, and wherein said sample has been frozen. In an other specific embodiment of the method, said sorting is performed by flow cytometry-based sorting. In an other specific embodiment of the method, said ligands are monoclonal antibodies.

In accordance with a second aspect of the present invention, there is also provided a method for monitoring thymic function in an individual comprising (a) obtaining a biological sample susceptible of containing T cells from the individual; (b) measuring the amount of T cells having a phenotype comprising at least the following four markers CD3⁺, CD4⁺, CD45RA⁺ and CD31^{hi} in the sample; (c) repeating steps (a) and (b) using the same type of biological sample as that obtained in step (a) but obtained from the individual at a subsequent point in time; and (d) comparing the amount of T cells measured in step (b) with that measured in step (c), thereby monitoring the progression or regression of thymic function in the individual. In a specific embodiment of the method, said phenotype further comprises at least the following two markers, namely CD27⁺ and CD62L⁺ or CCR7⁺. In an other specific embodiment of the method, said monitoring is for evaluating a therapy's efficacy. In an other specific embodiment of the method, said therapy is selected from the group consisting of vaccination, antiviral therapy, T cell reconstitutions, bone marrow transplantation and haematopoietic stem cell transplantation. In an other specific embodiment of the method, said monitoring is for evaluating a therapy's side effects. In an other specific embodiment of the method, said therapy is radiotherapy, chemotherapy, drug therapy. In an other specific embodiment of the method, said monitoring is for evaluating effects on thymic function of a disorder.

In an other specific embodiment of the methods, said individual is immunocompromised. In a more specific embodiment of the methods, said disorder is a hormonal or an endocrinal disorder.

In accordance with a third aspect of the present invention, there is also provided a method of diagnosing immune system dysfunction in an individual, comprising (a) measuring the amount of T cells having a phenotype comprising at least the following four markers CD3⁺, CD4⁺, CD45RA⁺ and CD31^{hi} in a biological sample from the individual; whereby an amount of said T cells is different from that found in a healthy individual of the same age is an indication that the individual has an immune system dysfunction. In a specific embodiment of the method, said phenotype further comprises at least the following two markers, namely CD27⁺ and CD62L⁺ or CCR7⁺. In an other specific embodiment of the method, said at least two additional markers comprise CD62L⁺, and wherein said sample has not been frozen. In an other specific embodiment of the method, said at least two additional markers comprise CCR7⁺, and wherein said sample has been frozen.

In an other specific embodiment of the methods of the present invention, said biological sample is whole or fractioned blood. In an other more specific embodiment of these methods, said biological sample is whole blood. In an other more specific embodiment of these methods, said CD31^{hi} marker identifies cells that are in the 40% higher percentile of cells in the biological sample in terms of CD31⁺ expression levels.

In accordance with a fourth aspect of the present invention, there is also provided a kit comprising at least four ligands, namely ligands directed to the following markers CD3⁺, CD4⁺, CD8⁺, CD45RA⁺ and CD31⁺. In a specific embodiment, the kit further comprises at least two further ligands, namely ligands directed to the following markers CD27⁺ and CD62L⁺ or CCR7⁺. In an other specific embodiment of the kit, said ligands are labelled. In an other specific embodiment of the kit, said ligands are monoclonal antibodies.

In accordance with a fifth aspect of the present invention, there is also provided a purified subpopulation of naive T cells having a phenotype comprising at least the following four markers CD3⁺, CD4⁺, CD45RA⁺, and CD31^{hi}. In an other specific embodiment of the purified subpopulation of naive T cells, the phenotype further comprises at least the following two markers CD27⁺ and CD62L⁺ or CCR7⁺. In an other specific embodiment of the purified subpopulation of naive T cells, the said CD31^{hi} identifies cells that are in the 40% higher percentile of cells in the biological sample in terms of CD31⁺ expression levels.

In accordance with a sixth aspect of the present invention, there is also provided a pharmaceutical composition comprising a purified subpopulation of naive T cells of the present invention and a pharmaceutically acceptable carrier.

In accordance with a seventh aspect of the present invention, there is also provided a method of using a purified subpopulation of naive T cells of the present invention for T cell immune reconstitutions in immunocompromised individuals.

As used herein the terminology “CD31^{h1} naive CD4 T cells” is meant to refer to CD4⁺ T cells having a phenotype comprising at least the following four markers CD3⁺, CD4⁺, CD45RA⁺ and CD31^{hi} In a preferred embodiment, the phenotype further comprises the markers CD27⁺, CD62L⁺ or CCR7^{+.}

As used herein the term “amount” refers to frequency and cell count.

As used herein the terminology “late stage thymocytes” refers to a cell that is about to exit thymus and has a phenotype that is close to that of a cell that has just exited thymus.

As used herein the terminology “naive T cell” is meant to refer to a cell that has not encountered an antigen. It comprises RTEs and other thymic emigrants having undergone many divisions. The terminology “naive CD4⁺ T cells” designate a population of cells that are heterogeneous in terms of phenotype.

As used herein the terminology “classical naive T cell” is meant to refer to CD45RA³⁰ CD62L⁺ (CD3⁺, CD4⁺ implied). However, it is important to note that there are many naive T cell phenotypes accepted in the scientific community. Indeed, CD45RA+CD27+ as well as CD45RA+CCR7+ are also widely used in the scientific literature.

As used herein the terminology “diseased individuals” is meant to refer to individuals with an immune system dysfunction.

As used herein the terminology “immune system dysfunction” refers to any clinical manifestation of subnormal or supra-normal numbers, activation levels, production rate and/or death rate of lymphocytes or other immune cells. Without being so limited, this terminology includes pathologies such as acute and chronic viral infections (HIV and AIDS, HCV, EBV, etc. . . ), autoimmune diseases (multiple sclerosis, Crohn's disease, autoimmune endocrine disorders (autoimmune polyglandular syndromes, Graves' disease, immune mediated diabetes, etc.) rheumatoid arthritis, systemic lupus erythematosus, etc.), cancers (all forms, before and after chemotherapy). A transient immune system dysfunction is also found in patients undergoing the immune reconstitution through bone marrow transplantation (BMT) and allogeneic haematopoietic stem cell transplantation (AHSCT) following myeloablative regimen treatment for haematological malignancies and possibly in other of the diseases mentioned above.

As used herein the terminology “ligand” refers to a compound or molecule able to specifically bind to a specific antigen found on an RTE identified by the present invention. Without being so limited, ligands encompassed by the present invention include monoclonal antibodies specific to antigens found on RTEs, as disclosed herein. Ligands of the present invention may advantageously be labelled to enable each antigen to be distinguishable from the others. Without being so limited, labels appropriate for the present invention include those used in the Examples along with any other fluorochromes than can be coupled to the monoclonal antibody structure (such as Alexa 350™, Alexa 488™, etc. . . ) and beads enabling by their composition and/or size their differentiation in cytomoters. Antibodies can further be coupled to magnetic beads or any other tool known in the art to facilitate the purification of ligand-bound CD4⁺ T cells.

As used herein the terminology “biological sample susceptible of containing T cells” refers to any biological sample from which T cells may be extracted. Without being so limited, this terminology refers to whole or fractioned blood (i.e density-gradient isolation of PBMCs), lymph nodes, spleen, bronchioalveolar washes, skin, intestine. In a preferred embodiment it refers to whole or fractioned blood.

As used herein the terminology “same type of biological sample” as used in descriptions of methods of present invention means that when the biological sample obtained at the first point in time is blood for instance, the sample obtained at the second point in time is also blood. This enables the measures obtained at each two point in time to be comparable.

As used herein the terminology “pharmaceutically acceptable carrier” refers to a solution, suspension, etc. prepared with commonly used excipients such as those described in Modern Pharmaceutics, 4th edition. Banker G S and Rhodes C T (eds) Marcel Dekker, N.Y., 2002.

As used herein the terminology “individual” refers to a human individual that is healthy or diseased.

As used herein, the terminology “thymic function” is meant to refer to the level of thymic activity, thus the quantity of output of recent T cells from the thymus to peripheral blood and/or secondary lymphoid organs and/or any other tissue containing lymphocytes.

As used herein the terminology “CD31^{hi}” refers to the phenotype of cells that are at least in the 80% higher percentile of cells in the tested biological sample in terms of CD31⁺ expression levels at their cell surface (as defined by number of molecules per cell) within “positivity” (as defined below). Preferably these cells are in the 70% higher percentile, more preferably 60%, even more preferably 50%, even more preferably 40%, even more preferably 30%, even more preferably 20% and most preferably 10%. The 80% higher percentile (CD31^{hi}) is the sum of the very high (vHi) and the intermediate expression level of CD31⁺. The CD31⁺ expression levels within “positivity” is calculated after having substracted the background. Indeed, in order to determine the level that defines CD31 vHi, intermediate, low and neg, the level of expression is split according to background. Indeed, in the present invention, the cells bear up to 6 fluorochome-coupled monoclonal antibodies. In addition, the array of fluorescence produced may “leak” within the detectors aimed at measuring the level of CD31. This creates a certain background level of fluorescence that is compensated (with standard flow cytometry compensation protocols using the FACSDiVa™ software) but remains present at low levels. With this in mind, a threshold of “positivity” is defined by standard flow cytometry comparison methods (i.e comparison to single stain and “fluorescence-minus-one” controls) as understood by a person of ordinary skill in the art. All cells with a fluorescence level above that threshold are then considered positive for the antigen. Thus, in Examples presented herein, cells in the lowest 50%, namely background levels, are considered negative for CD31 (CD31^{neg}). Within the CD31 positive fraction of cells (all cells with fluorescent levels above background), the level of CD31 expression was sub-defined in three subsets. Firstly, CD31low cells were determined to be within the 20% lowest fluorescent intensity levels of positivity. Secondly, CD31^{vHi} cells were defined to be within the highest 40% in fluorescent intensity level of positivity. The middle 40% is the third intermediate subset. Thus, cells referred to CD31hi naïve CD4 T cells are composed of both CD31vhi and the CD31 intermediate population.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 graphically identifies a sjTREC-rich population of CD4⁺ T cells. sjTREC frequencies were measured in various peripheral blood subpopulations from healthy individuals sorted by flow cytometry. a. Flow cytometry data where each dot represents a cell and shows its expression level of the corresponding fluorescence emission, which relates in turn to the expression level of the antigen indicated by the axis. This represents the gating strategy used for sorting CD31 high, low and negative CD3⁺CD4⁺CD8⁻CD45RA⁺CD62L⁺CD27⁺ cells. Following the selection of CD3⁺CD4⁺CD8⁻ (not shown), cells were gated according to their level of CD31 and CD27 expression. From this, each cell population (defined as “very high”, “low” and “neg”) was further gated according to CD45RA and CD62L expression. These cells were then purified through flow cytometry-based cell sorting. b. Effect of the levels of CD31 expression on sjTREC frequencies. After sorting fresh, Ficoll-isolated cells from 10 healthy individuals A to K (of indicated ages) according to panel a followed by the lysis of these cells, sjTRECs were quantified by nested real-time PCR. As previously decribed³, CD3γ was co-amplified with the sjTRECs and used as a housekeeping gene. All sjTREC quantifications were done in duplicates. All assays contained a minimum of 5000 cells, as determined by the CD3γ amplification. CD3⁺CD4⁺CD8⁻CD45RA⁺CCR7⁺CD27⁺ were also sorted according to their CD31 expression level in previously isolated and frozen cells from 4 healthy individuals L to O. sjTREC levels were assessed in the same manner as above. c. Enrichment of sjTREC levels in CD3⁺CD4⁺CD8⁻CD45RA⁺CD62L⁺CD27⁺CD31^{hi} compared to classical naive CD4⁺ T cells (CD3⁺CD4⁺CD8⁻CD45RA⁺CD62L⁺). Using the same method as in panel b, sjTRECs were quantified in total PBMCs, total CD4⁺ T cells (CD3⁺CD4⁺CD8⁻), naive CD4⁺ T cells (CD3⁺CD4⁺CD8⁻CD45RA⁺CD62L⁺) and CD31^{hi} naive CD4⁺ T cells (CD3⁺CD4⁺CD8⁻CD45RA⁺CD62L⁺CD27⁺CD31^{hi}). Represented here are means from 10 different healthy individuals with ages ranging from 24 to 58 yrs old, including C to F from panel b;

FIG. 2 graphically shows a comparison of sjTREC frequencies in thymocytes to CD31^{hi} naive CD4⁺ T cells. Following mechanical dissociation of pediatric thymic tissue, total thymocytes (TT) were sampled or stained and sorted in the following populations: CD4⁺CD8⁺(DP), CD3⁺CD4⁺CD8⁻CD1a⁺ (CD4 1a+), CD3⁺CD4⁺CD8⁻CD1a⁻CD45RA⁺CD62L⁺CD31⁺ (CD4 1a−), and CD3⁺CD4⁺CD8⁻CD45RA⁺CD62L⁺CD27⁺CD31⁺ (Thy31 CD4). Each of these subsets was assessed for sjTREC frequencies in duplicates. Represented here are the sjTREC frequency means of thymic tissue samples from 5 different donors (full histogram bars). The dashed histogram bar represents the mean sjTREC value for peripheral ^{CD31hi} naive CD4⁺ T cells (CD3⁺CD4⁺CD8⁻CD45RA⁺CD62L⁺CD27⁺CD31^{hi}) in adult PBMCs as presented in FIG. 1. The fold dilution is indicated in italic form;

FIG. 3 graphically shows the relationships between age, sjTRECs and immunophenotyping. In 20 healthy individuals from 28 to 59 years old, sjTREC quantification and % CD31^{hi} naive CD4⁺ T cells (CD3⁺CD4⁺CD8⁻CD45RA⁺CD62L⁺CD27⁺CD31^{hi}) in total PBMCs were assessed independently. a. Age-associated decrease in thymic function as measured by sjTREC quantification b. Age-associated decrease in thymic function as measured by immunophenotyping with CD31^{hi} naive CD4⁺ T cells. The percentage of CD3⁺CD4⁺CD8⁻CD45RA⁺CD62L⁺CD27⁺CD31^{hi} cells in PBMCs and the percentage of CD3⁺CD4⁺CD8⁻CD45RA⁺CD62L⁺ in PBMCs were measured by flow cytometric analysis. c. Two independent measures correlate. TREC quantification and immunophenotyping of CD31^{hi} naive CD4⁺ T cells (CD3⁺CD4⁺CD8⁻CD45RA⁺CD62L⁺CD27⁺CD31^{hi}) correlate with each other; and

FIG. 4 graphically illustrates the determination of the minimum requirements that identify RTEs. a. Cells were sorted as in FIG. 1, with the additional sorting of the subpopulations that do not take into account the expression of CD27, CD62L or either of them. CD3⁺CD4⁺CD8⁻CD45RA⁺CD31^{hi}, CD3⁺CD4⁺CD8⁻CD45RA⁺CD27⁺CD31^{hi}, CD3⁺CD4⁺CD8⁻CD45RA⁺CD62L⁺CD31^{hi} and CD3⁺CD4⁺CD8⁻CD45RA⁺CD62L⁺CD27⁺CD31^{hi} cells along with their CD31⁻ counterparts were sorted by flow cytometry and TREC levels were assessed in order to determine the TREC enrichment acquired with the use of CD62L and CD27 in healthy individuals. b. Correlations of TREC frequencies and age with the frequency of cells expressing different levels of CD31. As described in FIG. 3, PBMCs from 20 healthy individuals were stained with the RTE cocktail of mAbs and overall TREC frequencies were assessed. Here the correlation coefficients (pearson's coefficient) was compared with age and log transformed TREC levels to log transformed CD3⁺CD4⁺CD8⁻CD45RA⁺CD62L⁺CD27⁺CD31^{hi} (black circles) frequency of cells and CD3⁺CD4⁺CD8⁻CD45RA⁺CD62L⁺CD27⁺CD31^{low} (grey squares) frequency of cells in PBMCs, along with their CD31⁻ counterparts (open triangles).

### DESCRIPTION OF ILLUSTRATIVE EMBODIMENTS

This invention relates to the use of ligands such as specific monoclonal antibodies to evaluate the presence and/or amount of CD4⁺ T cells expressing 4 to 6 markers (CD3, CD4, CD45RA and CD31 (with or without CD62L or CCR7 and CD27: CD8 being negative i.e. It is widely accepted that, in peripheral blood, all cells bearing CD3 and CD4 do not bear CD8). as an indication of thymic function.

This invention particularly demonstrates that CD3⁺CD4⁺CD8⁻CD45RA⁺CD62L⁺CD27⁺CD31^{hi} and CD3⁺CD4⁺CD8⁻CD45RA⁺CCR7⁺CD27⁺CD31^{hi} are RTEs, thus indicative of thymic function, since they come directly from thymocytes exiting from thymus. Therefore, measuring frequencies and numbers of CD3⁺CD4⁺CD8⁻CD45RA⁺CD62L⁺CD27⁺CD31^{hi} or CD3⁺CD4⁺CD8⁻CD45RA⁺CCR7⁺CD27⁺CD31^{hi} cells constitutes a marker for evaluating thymic function and the biological status of its emigrants. The latter can be useful in the diagnostic or the follow-up of viral infection, vaccination, antiviral therapy and/or immune status (namely in HIV patients), graft, autoimmune disease, etc.

Color flow cytometry was used to sort surface antigens-bearing CD4⁺ T cells. A flow cytometer was used to sort and isolate CD3⁺CD4⁺CD8⁻CD45RA⁺CD62L⁺(or CCR7⁺) CD27⁺CD31⁺ cells with over 98% purity. Purity was verified by re-analysis of the sorted cells through the flow cytometer. Usually, over 95% purity is widely accepted in the scientific community. By using antibodies that identify these cells all in one step, a valid phenotype is ensured and experimental errors that can be induced by multi-step procedures are limited.

As it has been proposed that CD31 expression is lost following a post-thymic selection process, the present invention first sought to determine whether high CD31 expression levels in combination with naive CD4⁺ T cell markers (CD3 CD4 CD8 CD45RA CD62L and CD27), indicated a more recently exported CD4⁺ T cell from the thymus. To further elucidate if CD3⁺CD4⁺CD8⁻CD45RA⁺CD62L⁺CD27⁺CD31^{hi} cells were RTEs, namely as close as possible to exiting thymocytes, their sjTREC content was compared to those of late stage thymocytes. To verify whether CD3⁺CD4⁺CD8⁻CD45RA⁺CD62L⁺CD27⁺CD31^{hi} cells consistently decline with age, as does thymic function, their frequency in healthy individuals in various age groups was assessed. Finally, to demonstrate the accuracy of the present method to measure thymic function, thymic-derived recent CD4⁺ T cells, cell surface phenotyping of CD3⁺CD4⁺CD8⁻CD45RA⁺CD62L⁺CD27⁺CD31^{hi} was compared to sjTREC assessment in PBMCs.

The present invention is illustrated in further details by the following non-limiting examples.

### EXAMPLE 1

### Isolation of PBMCs from Blood Samples

Blood and leukapheresis samples were obtained from healthy adult donors. Samples were obtained with the informed consent of the subjects and according to the guidelines of the bioethical committee of Centre hospitalier de l'Université de Montreal (CHUM) and McGill University Health Center (MUHC).

PBMCs were obtained from blood samples using density gradient sedimentation with Ficoll Paque Plus™ (Amersheam Bioscience). Cells were washed twice with PBS 2% FCS before staining with mAbs for flow cytometry.

### EXAMPLE 2

### Isolation of Thymocytes from Thymus Samples

Pediatric thymic tissues were sampled from children undergoing elective cardiac surgery from Saint-Justine Hospital as well as CHEO, with the informed consent of the child's parents and according to the guidelines of the bioethical committee of Centre hospitalier de l'Université de Montreal (CHUM) and Research Ethics Board of the Children's Hospital of Eastern Ontario. Following mechanical dissociation of thymic tissue using cell strainers (Falcon), isolated thymocytes were harvested and stained.

### EXAMPLE 3

### Cell Surface Phenotying and Sorting by Flow Cytometry

PBMCs. Freshly isolated cells were stained simultaneously with the following mAbs : FITC labeled anti-CD31(BD Pharmingen), PE labeled anti-CD27(BD Pharmingen), PECy5 labeled anti-CD45RA(BD Pharmingen), PECy7 labeled anti-CD3(BD Pharmingen), APC labeled anti-CD62L(BD Pharmingen), APC-Cy7 labeled anti-CD4 (BD Pharmingen) and ECD labeled anti-CD8 (Coulter). In the event of phenotyping, the cells were analysed by 7-color flow cytometry with the FACS LSR II™ (Becton Dickinson) using FACSDiVa™ (Becton Dickinson) software. Previously isolated cells and frozen cells were thawed and stained simultaneously with the following mAbs: PE labeled anti-CD31 (BD Pharmingen), PECy5 labeled anti-CD45RA(BD Pharmingen), PECy7 labeled anti-CCR7 (BD Pharmingen), APC labeled anti-CD3(BD Pharmingen), APC-Cy7 labeled anti-CD27 (BD Pharmingen) Alexa 700 labeled anti-CD4 (BD Pharmingen), and ECD labeled anti-CD8 (Coulter). Seven-color cell sorting was performed using the FACSAria™ (Becton Dickinson).

Thymus. Freshly isolated thymocytes were stained with, in addition to the mAbs listed above, PE-labeled anti-CD1a (BD Pharmingen). Cell sorting was performed using the FACSAria™ with the FACSDiVa™ software.

All sorted samples were centrifuged and resuspended in lysis buffer for sjTREC analysis.

### EXAMPLE 4

### sjTREC Quantification

Primers specific for the sjTREC (δRec-ΨJα) and the human CD3γ-chain gene were defined on the human germline sequence (Genebank accession numbers AE00061 and X06026; Table 1 below). Parallel quantification of sjTREC together with the CD3γ amplicon was performed for each sample using the LightCycler™ technology (Roche Diagnostics). All samples were resuspended in lysis buffer: Tween-20™ (0.05%), NP-40 (0.05%), TRIS-HCl pH 8.0 (0.1 M) and Proteinase K (100 μg/mL) and incubated for 30 min at 56° C. and then 15 min at 98° C. Multiplex PCR amplification was performed for sjTREC together with the CD3γ chain, in 100 μL (10 minute initial denaturation at 95° C., 30 seconds at 95° C., 30 seconds at 60° C., 2 minutes at 72° C. for 22 cycles) using outer 3′/5′ primer pairs. PCR conditions in the LightCycler™ experiments, performed on 1/100^{th} of the initial PCR, were: 1 minute initial denaturation at 95° C., 1 second at 95° C., 10 seconds at 60° C., 15 seconds at 72° C. for 40 cycles. Fluorescence measurements with probes (were performed at the end of the elongation steps. TRECs and CD3γ LightCycler™ quantifications were performed in independent experiments, but on the same first-round serially diluted standard curve. This highly sensitive nested quantitative PCR assay allows the detection of one copy out of 10⁵ cells for each DNA circle. The sjTRECs were quantified in duplicates for all studied samples.

TABLE 1

OLIGONUCLEOTIDES USED FOR REAL TIME PCR
QUANTIFICATIONS

PCR primers

| | |
|---|---|
| sj-out5 | |
| 5′-CTCTCCTATCTCTGCTCTGAA-3′ | (SEQ ID NO: 1) |
| sj-out3 | |
| 5′-ACTCACTTTTCCGAGGCTGA-3′ | (SEQ ID NO: 2) |
| sj-in5 | |
| 5′-CCTCTGTCAACAAAGGTGAT-3′ | (SEQ ID NO: 3) |
| sj-in3 | |
| 5′-GTGCTGGCATCAGAGTGTGT-3′ | (SEQ ID NO: 4) |
| CD3-out5 | |
| 5′-ACTGACATGGAACAGGGGAAG-3′ | (SEQ ID NO: 5) |
| CD3-out3 | |
| 5′-CCAGCTCTGAAGTAGGGAACATAT-3′ | (SEQ ID NO: 6) |
| CD3-in5 | |
| 5′-GGCTATCATTCTTCTTCAAGGT-3′ | (SEQ ID NO: 7) |
| CD3-in3 | |
| 5′-CCTCTCTTCAGCCATTTAAGTA-3′ | (SEQ ID NO: 8) |

LightCycler ™ probes

| | |
|---|---|
| sj-P1 | |
| 5′-AATAAGTTCAGCCCTCCATGTCACACTf-3′ | (SEQ ID NO: 9) |
| sj-P2 | |
| 5′-XTGTTTTCCATCCTGGGGAGTGTTTCAp-3′ | (SEQ ID NO: 10) |
| CD3-P1 | |
| 5′-GGCTGAAGGTTAGGGATACCAATATTCCTGT | (SEQ ID NO: 11) |
| CTCf-3′ | |
| CD3-P2 | |
| 5′-XCTAGTGATGGGCTCTTCCCTTGAGCCCTTC | (SEQ ID NO: 12) |
| p-3′ | |

Statistics. Statistical analysis (two-tailed paired Student t-test, Pearson correlation test, r, and p values) was performed using the Vassar college website and MS Excel. An r value ≧0.5 or ≦−0.5, and a p value ≦0.01, were considered significant.

### EXAMPLE 5

### Determination of sjTREC Levels in CD3⁺CD4⁺CD8⁻CD45RA⁺CD62L⁺CD27⁺CD31^{hi} Cells

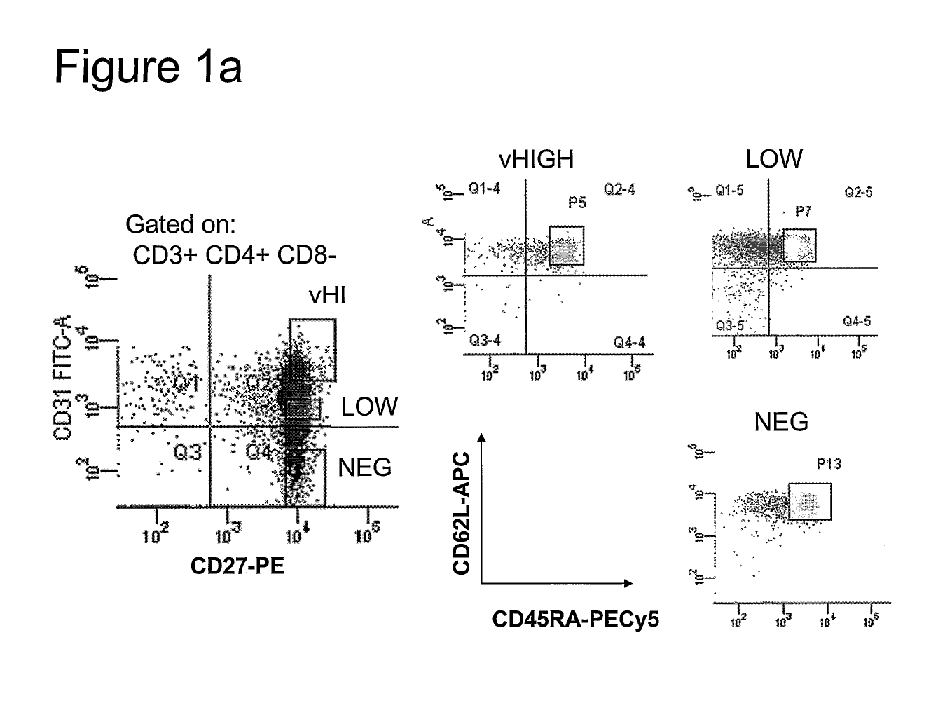

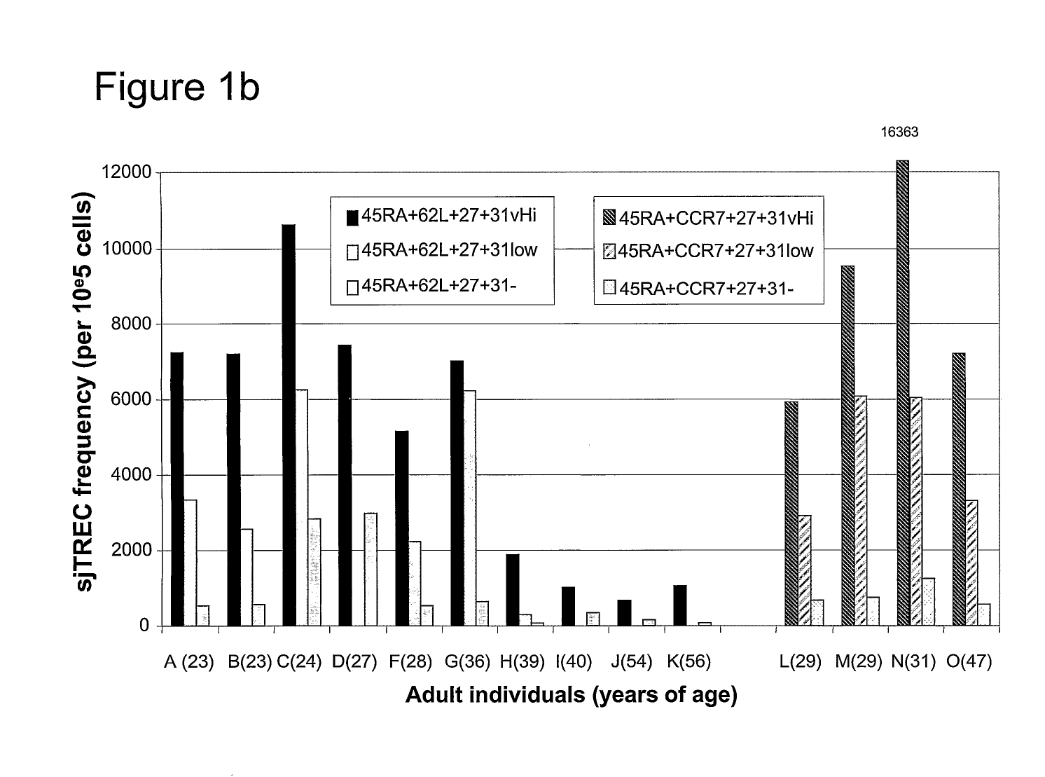

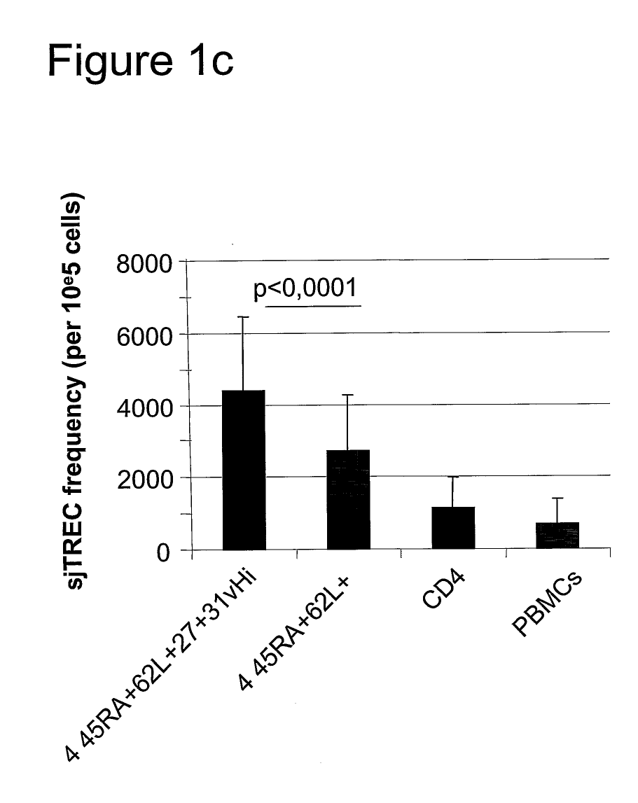

As cells divide, TRECs are diluted out. Thus, TREC frequencies can reveal the number of rounds of proliferation a population has undergone and, to some extent, the developmental “age” since thymic exportation of the cells within that population. With this in mind, the correlation of CD31 expression levels on the CD4⁺T cell maturity was determined by assessing sjTREC levels in FACS isolated naive CD4⁺ T cells (CD45RA⁺CD62L⁺) cells, separated by CD31^{hi}, CD31^{lOw} or CD31⁻. Using 7-color flow cytometry, CD3⁺CD4⁺CD8⁻CD45RA⁺CD62L⁺CD27⁺CD31^{hi}, (from now on referred to as CD31^{hi} naive CD4 T cells), CD3⁺CD4⁺CD8⁻CD45RA⁺CD62L⁺CD27⁺CD31^{low} (CD31^{low} naive CD4⁺ T cells) and CD3⁺CD4⁺CD8⁻CD45RA⁺CD62L⁺CD27⁺CD31⁻ cells (CD31⁻ naive CD4⁺ T cells) were analysed and sorted. As depicted in FIG. 1a, these phenotypes were determined: “CD31^{vHi}” as the highest fifth of CD31 (expression level which corresponds to the 40% higher percentile within positivity), the “CD31^{low}” as the 20% lowest percentile and the CD31^{intermediate} as the middle 40% percentile (not shown). Reanalysis of the sorted samples revealed a greater than 98% purity of each population (data not shown). Real-time PCR-based sjTREC quantification in these subpopulations showed on average 10-fold significantly greater sjTREC levels (Paired Student t-test=0.001) in CD31^{hi} naive CD4⁺ T cells (n=10 mean=4934 sjTRECs/10e5 cells range: 662−10655) when compared to CD31⁻ naive CD4⁺ T cells (mean=865 range: 68-2954) in the same individuals (FIG. 1b). Furthermore, CD31^{low} naive CD4⁺ T cells possess 5-fold lower sjTREC levels (mean=3484 range: 311-6269) than in their CD31^{hi} counterparts (Paired Student T-test=0.005) (FIG. 1b). This confirms the existence of two main CD4⁺ naive T cell compartments: a sjTREC-low naive CD4⁺ T cell compartment as defined by CD31⁻ cells, a population having undergone extensive proliferation, and a highly sjTREC-enriched CD31^{hi} CD4⁺ naive T cell compartment, likely recently exited thymic emigrants. A population of naive CD4⁺ T cells with sjTREC levels found in between CD31^{hi} and CD31⁻ CD4 naive T cells was also identified, namely the CD31^{lOw}, most probably a population that represents the developmental transition of one to the other, thus, not recent thymic emigrants. As this assay is intended to be used on fresh and frozen samples, and that CD62L is shedded upon thawing of cells, the use of another known marker of naive T cells, CCR7, was attempted on frozen samples. As expected, replacement of CD62L by CCR7 did not change the differences in sjTREC frequency in CD31 high, low and negative cells. The present invention thus considers swapping CD62L with CCR7 as a viable option when working with frozen cell samples, and CD3⁺CD4⁺CD8⁻CD45RA⁺CCR7⁺CD27⁺CD31^{hi} cells can also be considered as RTEs.

Interestingly, overall sjTREC levels in CD31^{hi} CD4 naive T cell as well as CD31⁻ CD4 naive T cell significantly decrease with age (respectively r=−0.850, r=−0.537). On the other hand, TREC levels in CD31^{lOw} naive CD4 T cells did not significantly inversely correlate with age (r=−0.226). Within another group of healthy individuals, it was shown that using CD31 in naive CD4 T cells has a significant advantage over the classical naive phenotype (CD45RA⁺CD62L⁺) in the identification of newly emigrated T cells of thymic origin. Indeed, CD31^{hi} naive CD4 T cells possess on average 1,4-fold greater sjTREC levels (mean=4669 range: 1212-7433) than overall naive CD4 T cells (mean=3400 range: 521-5154) (Paired Student T-test=0.002) (FIG. 1c).

### EXAMPLE 6

### Comparison of TREC Levels of CD31^{hi} naive CD4 T Cells with Those of Exiting Thymocytes

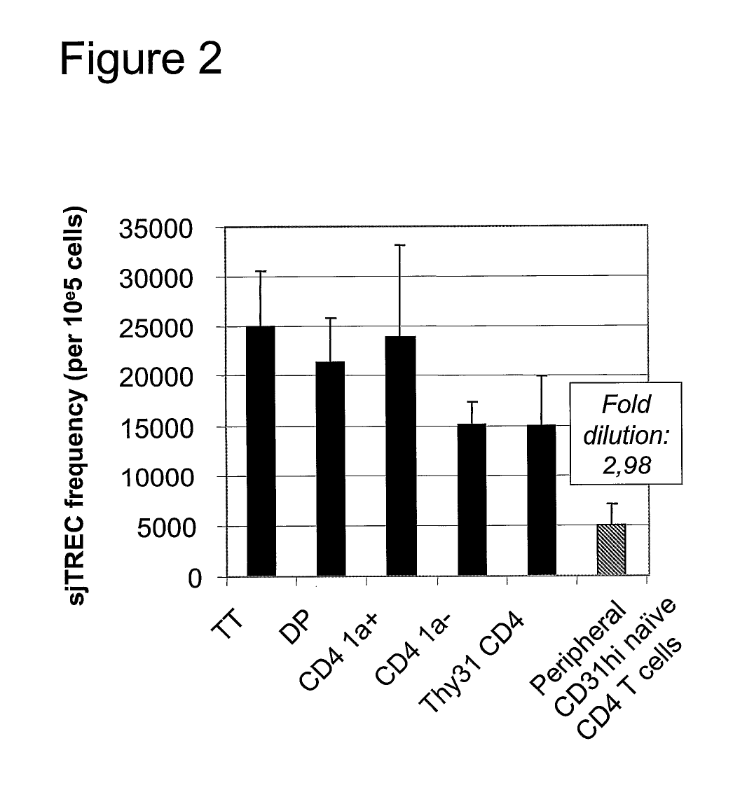

sjTRECs are mainly produced in thymocytes, more specifically within the CD4+CD8+(DP) cells⁴, when the TCRD locus is excised from the TCRA. In order to verify what sjTREC frequencies to expect in an RTE phenotype, sjTREC levels were assessed in post-DP thymocytes subpopulations. To do so, thymocytes from pediatric thymii (n=5) were sorted and sjTREC levels quantified (FIG. 2). Similarly to previously described data⁴, DP thymocytes have a mean 20 621sjTREC/10e5 cells (range: 17123-27904). sjTREC levels are maintained as they mature into earlier single positive CD1a⁺ (mean=23801 range:15173-37662) and progressively decline into late single positive CD1a− thymocytes (mean=15047 range: 11873-18903), likely a consequence of previously described post-selection cell divisions⁵. Interestingly, CD31^{hi} naive CD4 T cell phenotype was found in the thymus (referred to herein as “Thy31 CD4”). Furthermore, TREC levels within the Thy31 CD4 cells (mean=14909 range: 10610-21600) were very similar to those of late single positive cells (i.e. 15047), demonstrating developmental similarities between these cells. When comparing the sjTREC levels of CD1a− and Thy31 CD4cells with those within peripheral CD31^{hi} naive CD4 T cells, a 3-fold difference was found. As sjTREC are diluted by half with each cell division, it appears that a mean 1,5 rounds of cell division separate peripheral CD31^{hi} naive CD4 T cells from exiting/late thymocytes (i.e. CD1a− and Thy31CD4). The fact that CD31^{hi} naive CD4 T cells possess sjTREC levels similar to those of late thymocytes strongly indicates this phenotype represents an RTE-like population.

### EXAMPLE 7

### Determination of CD31^{hi} Naive CD4 T Cells Levels Modification with Age

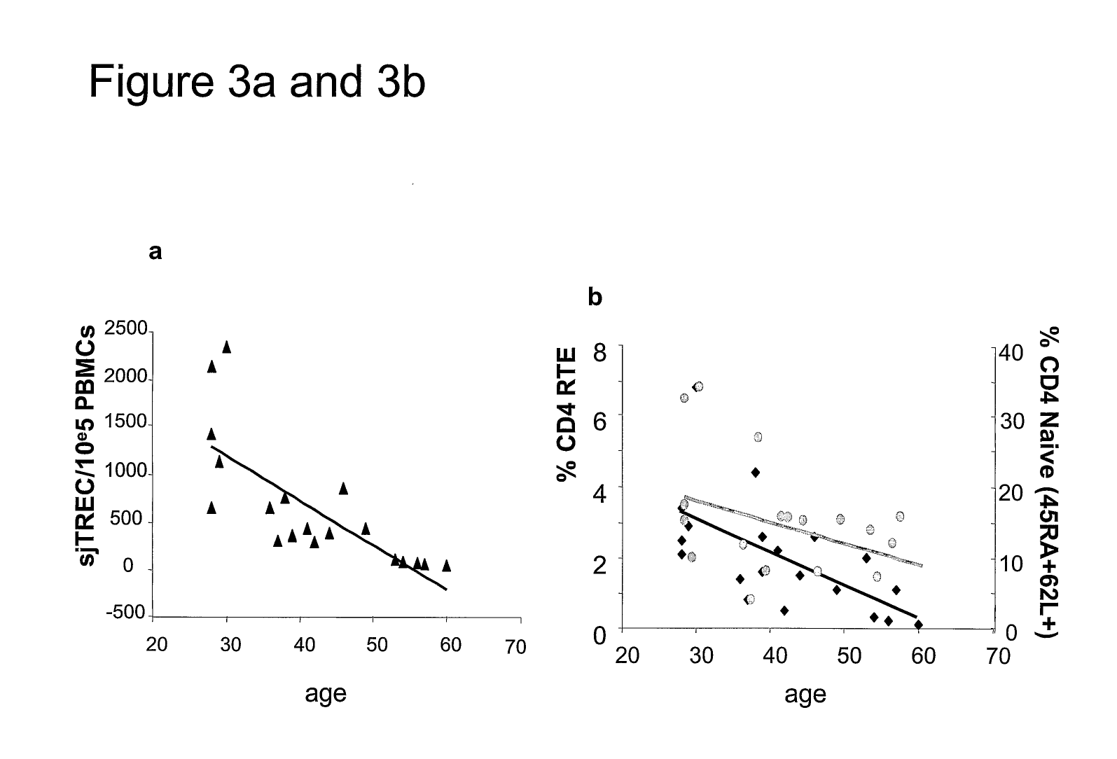

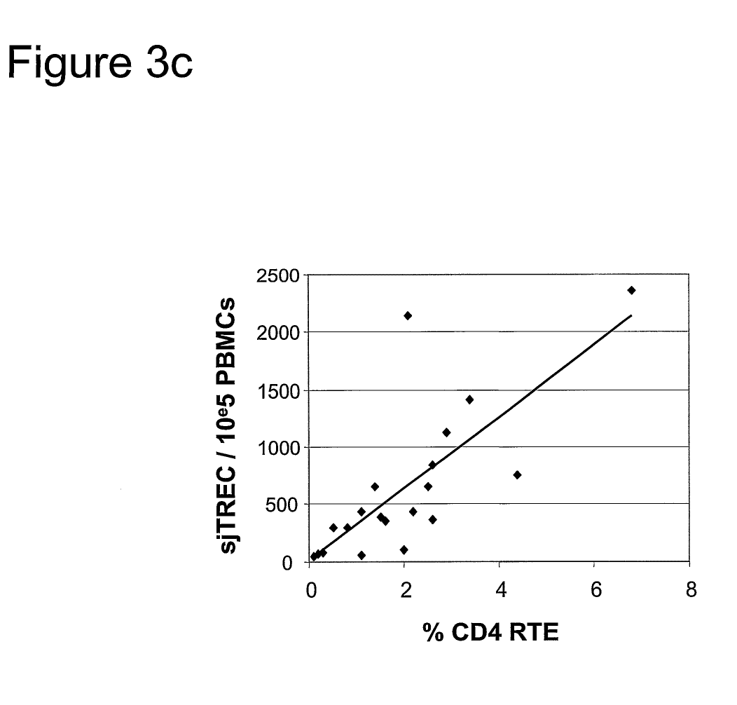

In order to verify if these cells were also consistent with the attribute of age decline of thymic function, sjTREC and CD31^{hi} naive CD4 T cells frequency in 20 healthy adult individuals of ages 23 to 60 years old were separately measured. As expected, sjTREC frequencies inversely correlated with age (r=−0.743 p=0.0002)(FIG. 3a). In accordance to this, cell surface phenotyping revealed frequencies varying from 0.1 to 6.8% of CD31^{hi} naive CD4 T cells in total PBMC with a significant age-decline (r=−0.613 p=0.003), unlike the classical CD4 naive T cell phenotype (r=−0.469 p=0.03) (FIG. 3b). In addition, both sjTREC and cell surface phenotyping methods yielded similar results within each individual, as both values strongly correlate (r=−0.756 p<0.0001), indicating the reliability of the phenotyping method in measuring thymic function (FIG. 3c). Overall, it was demonstrated that CD31^{hi} naive CD4 T cells represent RTEs in a more coherent manner than the classical naive T cells phenotype, and compare to results obtained using the TREC method.

### EXAMPLE 8

### Determination of the Minimal Phenotype to Represent RTEs

Although the characterization of cells bearing the complete set of naive phenotype surface markers (CD3⁺CD4⁺CD8⁻CD45RA⁺CD62L⁺CD27⁺ or CD3⁺CD4⁺CD8⁻CD45RA⁺CCR7⁺CD27⁺) with high expression of CD31 will identify a pure population of naive CD4 T cells closest to exiting thymocytes, it is possible to combine CD3, CD4, CD45RA and CD31 to identify recent thymic emigrants in healthy individuals.

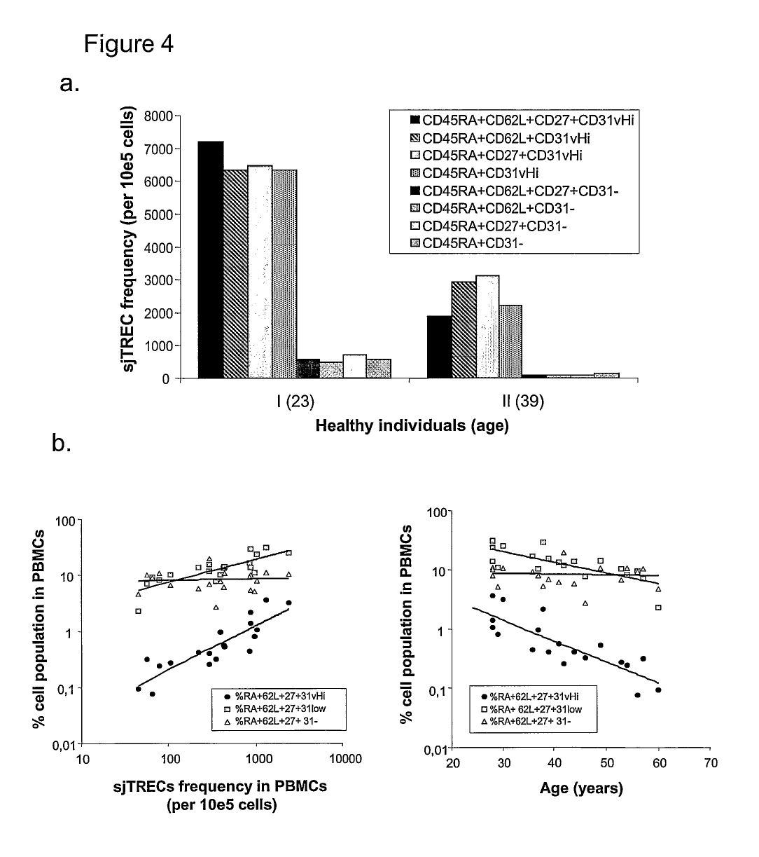

Indeed, naive CD4 T cells in healthy human adults are over 98% CD45RA+, thus the sole use of CD45RA is needed to isolate them. The addition of CD31 is key for separating early naive T cells (RTE) to more mature naive T cells (non-RTE). As demonstrated in FIG. 4a, sorted cells bearing CD3⁺CD4⁺CD45RA⁺CD31^{hi} from healthy human adults harbor TREC levels similar to those of CD3⁺CD4⁺CD45RA⁺CD62L⁺CD27⁺CD31^{hi} cells (as well as CD3⁺CD4⁺CD45RA⁺CD62L⁺CD31^{hi} and CD3⁺CD4⁺CD45RA⁺CD27⁺CD31^{hi} cells). Also, their CD31⁻ counterparts have equally less TRECs for all definition of naive T cell populations. Of note, although this is true in healthy individuals, in pathological situations, expansion of naive-like (CD45RA+) memory cells may hinder the assessment of a pure naive T cell population. According to DeRosa et al., the CD45RA⁺ phenotype used to identify naive cells is subject to artifacts due to the inclusion of memory T cells within the designated subset. They conclude that to overcome this, a minimum of three differentiation markers is required to consistently enumerate naive T cells with more than 95% accuracy, especially in pathological situations. Thus, the use of CD3, CD4, CD45RA and CD31 the minimum required to identify RTEs in healthy individuals. However, in clinical settings, it is strongly suggested that CD3, CD4, CD45RA, CD27, CD62L (or CCR7) and CD31 all be used to enumerate and characterize RTEs, as this will more specifically identify antigen-naive CD4 T cells having recently exited the thymus.

Through immune phenotyping of PBMCs from 20 healthy individuals, the correlations of CD3⁺CD4⁺CD45RA⁺CD62L⁺CD27⁺CD31^{hi}, CD3⁺CD4⁺CD45RA⁺CD62L⁺CD27⁺CD31^{low} and CD3⁺CD4⁺CD45RA⁺CD62L⁺CD27⁺CD31⁻ cells was compared with age and blood TREC levels. Contrarily to CD3⁺CD4⁺CD45RA⁺CD62L⁺CD27⁺CD31⁻, CD3⁺CD4⁺CD45RA⁺CD62L⁺CD27⁺CD31^{low} cells decline with age, but not as significantly as CD3⁺CD4⁺CD45RA⁺CD62L⁺CD27⁺CD31^{hi} cells (respectively, r =0.111 p=n.s, r=−0.746 p<0.02, r =−0.836 p<0.005). Furthermore, the frequency of CD3⁺CD4⁺CD45RA⁺CD62L⁺CD27⁺CD31^{low} cells in PBMCs correlates positively to sjTREC frequency, but not as tightly as CD3⁺CD4⁺CD45RA⁺CD62L⁺CD27⁺CD31^{hi} cells. It is thus to be concluded that CD3⁺CD4⁺CD45RA⁺CD62L⁺CD27⁺CD31^{low} cells although regulated similarly to CD3⁺CD4⁺CD45RA⁺CD62L⁺CD27⁺CD31^{hi} cells do not display as significantly all the characteristics common to thymic activity and consequently, its emigrants. With this in mind, through the exclusion of CD31 low CD4 naive T cells in the overall CD31 positive cells, the use of the highest 80%, preferably 70%, more preferably 60%, more preferably 50%, more preferably 40%, more preferably 30%, more preferably 20%, more preferably 10%, CD31 expression level in naive CD4⁺ T cells is the most representative phenotype of recent thymic emigrants.

Taken together, resulted presented herein show that naive CD4⁺ T cells positive for the CD31 surface marker highly expressed represent naive T cells with developmental proximity to the thymus. In healthy individuals, CD3⁺CD4⁺CD45RA⁺ is acceptable to identify naive CD4 T cells, and the addition of CD31 to this phenotype permits an enrichment in TREC levels. Thus, the use of antibodies, or other useful ligands if any, that characterize cells bearing CD3⁺CD4⁺CD45RA⁺CD31^{hi} is the minimum requirement to identify CD4 naive T cells of thymic developmental proximity in healthy individuals. However, the use of CD3⁺CD4⁺CD45RA⁺CD62L⁺CD27⁺CD31^{hi} remains the most accurate phenotype to identify RTE as this profile of cells will limit any possible caveats due to the expansion of other non-RTE cells with a “naive-like” phenotype, as defined by the minimum phenotypical requirement Indeed,, the use of many markers circumvents any inherent difficulties of the identification of specific cell subsets commonly encountered in pathological situations.

The RTE phenotyping and sorting has many advantages over TREC quantification and can be useful in a variety of clinical and research-oriented settings:

It is a simple, one-step non-invasive method to measure thymic function;

Using a phenotype that encompasses 7 markers as disclosed herein to define RTE cells, ensures that these cells are the most naive (not having encountered antigen), which is essential in immune dysfunctions and deficiencies where highly activated and differentiated T cell are present in greater number (i.e. HIV);

In contrast to TREC quantification, a highly controversial method of thymic function assessment in HIV infected individuals⁶, this method enables isolation of live cells with minimal manipulation. Indeed, circulating blood cells are used and because they are not lysed, they can be re-used or transferred into a patient.

Thus, treatment of a patient in need for an infusion of naive T cells is contemplated, with a view of restoring or stimulating the immune system. Further, diagnostic and follow up of diseases characterized by an immune system dysfunction (autoimmune diseases, Acute Immunodeficiency Syndrome, cancer, etc.) is also contemplated. Evaluation of immune therapy efficacy (vaccines including prophylactic vaccines on normal individuals, bone marrow transplantation, etc.) is an also aspect of this invention.

Research and development in the context of the thymic differentiation, and biology of recent thymic emigrants will also benefit from the present method.

A kit comprising four to six markers is an aspect of this invention. The ligands are preferably monospecific antibodies conjugated to different colored members, which enables the identification of the phenotypic surface markers.

Such a phenotyping method and kit will be of great use in quantification and characterization of thymic output in various pathological settings, such as immunodeficiency diseases, as well as assessing the effects of their treatments. In addition, further knowledge of the biological niche of RTE will also allow the development of novel therapies that will maintain the RTE population and favor the generation of new T cells. In addition, the production and preservation of a diverse T cell repertoire will assist in the development of successful vaccination therapies in contexts such as HIV, where thymic function is severely impaired. Overall, the method described here enables a one-step identification of the CD3⁺CD4⁺CD8⁻CD45RA⁺CD62L⁺CD27⁺CD31^{hi} cell phenotype, a reliable and accurate representation of RTEs, which is in turn a key component in understanding and treating immunological disorders

Although the present invention has been described hereinabove by way of specific embodiments thereof, it can be modified, without departing from the spirit and nature of the subject invention as defined in the appended claims.

1. A Method of purifying a subpopulation of naive T cells that have recently emigrated from the thymus, comprising (a) contacting a biological sample susceptible of containing T cells with at least four different ligands, namely ligands directed to the markers CD3, CD4, CD45RA, and CD31; (b) sorting the cells so as to recover T cells having a phenotype comprising at least the following four markers CD3⁺, CD4⁺, CD45RA⁺ and CD31^{hi}, whereby a subpopulation of naive T cells is purified.

2. The method of claim 1, wherein said contacting step comprises contacting the sample with at least two additional ligands, namely ligands directed to the markers CD27⁺ and CD62L⁺ or CCR7⁺.

3. The method of claim 2, wherein said at least two additional ligands comprise a CD62L ligand, and wherein said sample has not been frozen.

4. The method of claim 2, wherein said at least two additional ligands comprise a CCR7 ligand, and wherein said sample has been frozen.

5. The method of claim 1, wherein said sorting is performed by flow cytometry-based sorting.

6. The method as recited in any one of claims 1, wherein said ligands are monoclonal antibodies.

7. A Method for monitoring thymic function in an individual comprising (a) obtaining a biological sample susceptible of containing T cells from the individual; (b) measuring the amount of T cells having a phenotype comprising at least the following four markers CD3⁺, CD4⁺, CD45RA⁺ and CD31^{hi} in the sample; (c) repeating steps (a) and (b) using the same type of biological sample as that obtained in step (a) but obtained from the individual at a subsequent point in time; and (d) comparing the amount of T cells measured in step (b) with that measured in step (c), thereby monitoring the progression or regression of thymic function in the individual.

8. The method of claim 7, wherein said phenotype further comprises at least the following two markers, namely CD27⁺ and CD62L⁺ or CCR7⁺.

9. The method of claims 7, wherein said monitoring is for evaluating a therapy's efficacy.

10. The method of claim 9, wherein said therapy is selected from the group consisting of vaccination, antiviral therapy, T cell reconstitutions, bone marrow transplantation and haematopoietic stem cell transplantation.

11. The method of claims 7, wherein said monitoring is for evaluating a therapy's side effects.

12. The method of claim 11, wherein said therapy is radiotherapy, chemotherapy, drug therapy.

13. The method as recited in any one of claims 7, wherein said monitoring is for evaluating effects on thymic function of a disorder.

14. The method of claim 7, wherein said individual is immunocompromised.

15. The method of claim 13, wherein said disorder is a hormonal or an endocrinal disorder.

16. A method of diagnosing immune system dysfunction in an individual, comprising (a) measuring the amount of T cells having a phenotype comprising at least the following four markers CD3⁺, CD4⁺, CD45RA⁺ and CD31^{hi} in a biological sample from the individual; whereby an amount of said T cells is different from that found in a healthy individual of the same age is an indication that the individual has an immune system dysfunction.

17. The method of claim 16, wherein said phenotype further comprises at least the following two markers, namely CD27⁺ and CD62L⁺ or CCR7⁺.

18. The method of claim 8, wherein said at least two additional markers comprise CD62L⁺, and wherein said sample has not been frozen.

19. The method of claim 8, wherein said at least two additional markers comprise CCR7⁺, and wherein said sample has been frozen.

20. The method of claim 1, wherein said biological sample is whole or fractioned blood.

21. The method of claim 1, wherein said biological sample is whole blood.

22. The method of claims 1, wherein said CD31^{hi} marker identifies cells that are in the 40% higher percentile of cells in the biological sample in terms of CD31⁺ expression levels.

23. A kit comprising at least four ligands, namely ligands directed to the following markers CD3⁺, CD4⁺, CD8⁺, CD45RA⁺ and CD31⁺.

24. The kit of claim 23, further comprising at least two further ligands, namely ligands directed to the following markers CD27⁺ and CD62L⁺ or CCR7⁺.

25. The kit of claim 23, wherein said ligands are labelled.

26. The kit of claims 23, wherein said ligands are monoclonal antibodies.

27. A purified subpopulation of naive T cells having a phenotype comprising at least the following four markers CD3⁺, CD4⁺, CD45RA⁺, and CD31^{hi}.

28. The purified subpopulation of naive T cells of claim 27, wherein the phenotype further comprises at least the following two markers CD27⁺ and CD62L⁺ or CCR7⁺.

29. The purified subpopulation of naive T cells of claim 27, wherein said CD31^{hi} identifies cells that are in the 40% higher percentile of cells in a biological sample containing the purified subpopulation in terms of CD31⁺ expression levels.

30. A pharmaceutical composition comprising the purified subpopulation of naive T cells of claim 27 and a pharmaceutically acceptable carrier.

31. A method of using the purified subpopulation of naive T cells of claim 27 for T cell immune reconstitutions in immunocompromised individuals.
